(19) United States
(12) Patent Application Publication
(10) Pub. No.: US 2018/0280000 A1
(43) Pub. Date: Oct. 4, 2018

(54) ULTRASONIC PROBE, ULTRASONIC DIAGNOSTIC APPARATUS, AND ULTRASONIC DIAGNOSTIC ASSISTANCE METHOD

(71) Applicant: Canon Medical Systems Corporation, Otawara-shi (JP)

(72) Inventors: Yuki TAMANO, Nasushiobara (JP); Taketoshi NAGAI, Nasushiobara (JP); Yasuhiro TAKAHASHI, Nasushiobara (JP)

(73) Assignee: Canon Medical Systems Corporation, Otawara-shi (JP)

(21) Appl. No.: 15/928,762

(22) Filed: Mar. 22, 2018

(30) Foreign Application Priority Data
Mar. 31, 2017 (JP) 2017-071423
Mar. 15, 2018 (JP) 2018-047721

(51) Int. Cl. A61B 8/00

(52) U.S. Cl. CPC A61B 8/54; A61B 8/56

(57) ABSTRACT

According to one embodiment, an ultrasonic probe includes a plurality of ultrasonic transducers and a calculation circuitry. The calculation circuitry calculates part of delay data relating to a delay time which is set in each of the ultrasonic transducers, before transmission of ultrasonic waves.

### CROSS-REFERENCE TO RELATED APPLICATIONS

This application is based upon and claims the benefit of priority from the prior Japanese Patent Application No. 2017-071423, filed Mar. 31, 2017, and 2018-047721, filed Mar. 15, 2018, the entire contents of all of which are incorporated herein by reference.

### FIELD

Embodiments described herein relate generally to an ultrasonic probe, an ultrasonic diagnostic apparatus, and an ultrasonic diagnostic assistance method.

### BACKGROUND

An ultrasonic diagnostic apparatus transmits ultrasonic waves to a subject (patient) and receives reflective waves (echo) from the inside of the subject to generate an image of the inside of the subject. Recently, two-dimensional array type ultrasonic probes have been mainly used.

A two-dimensional array probe includes a large number of ultrasonic transducers (also referred to as elements) that are two-dimensionally arranged in the form of a grid, and thus it is difficult for the ultrasonic diagnostic apparatus body to directly drive all the elements to control transmission/reception of ultrasonic waves.

Therefore, the ultrasonic wave probe is provided inside with a specific IC (e.g., Application Specific Integrated Circuit: ASIC) configured to divide all the elements into sub-arrays each including some elements and perform partial delay calculation for each sub-array.

Setting of a delay time relating to each element in each of the sub-arrays needs to be performed during a time from an end of a reception period of echo to the next transmission timing of ultrasonic waves, which is referred to as a blanking period. Specifically, in the ultrasonic diagnostic apparatus body, it is necessary to calculate delay control data (delay data) relating to a delay time of transmission/reception of ultrasonic waves on a transmission/reception rate basis, transfer the delay data to the ultrasonic probe within the blanking period, and complete the setting of the delay time in the ultrasonic probe.

With a recent increase in the number of channel elements in a two-dimensional array probe, the amount of delay data to be transferred will increase. Therefore, it takes time to transfer and set delay data of transmission/reception delay, and thus a problem that transmission and setting of the delay data cannot be completed within a blanking period is estimated. For example, time has passed a blanking period in midstream of transfer of delay data.

As a solution to the problem, a means to increase the clock frequency of an input interface (input IF) of an ultrasonic probe may be considered; however, this is undesirable, because such a means causes an increase in power consumption and heat generation in the ultrasonic probe. A means to extend the setting period may also be considered; however, it causes a decrease in frame rate. Furthermore, a technique of preliminarily saving delay data in an ultrasonic probe may also be considered; however, since the amounts of data of delay data in a frame sequence of a two-dimensional array probe (in particular, in a simultaneous mode) are significantly large, it is necessary to prepare a large-scale memory, and thus it is not realistic from the viewpoints of the dimensional area of its substrate, power consumption, and costs. In addition, it is necessary to secure time when saving delay data, and thus the responsiveness degrades during transfer of vast amounts of delay data.

### BRIEF DESCRIPTION OF THE DRAWINGS

FIG. 1 is a block diagram showing the configuration of an ultrasonic diagnostic apparatus according to a first embodiment.

FIG. 2 is a diagram showing details of an ultrasonic probe.

FIG. 3 is a conceptual diagram showing an arrangement of two-dimensionally arrayed elements.

FIG. 4 is a diagram for illustrating calculation of entire fine transmission delay data.

FIG. 5 is a conceptual diagram of a blanking period according to the present embodiment.

FIG. 6 is a diagram showing a first allocation example of a calculation process of delay data.

FIG. 7 is a diagram showing a second allocation example of the reception period of delay data.

FIG. 8 is a diagram showing a third allocation example of the reception period of delay data.

FIG. 9 is a diagram showing a fourth allocation example of the reception period of delay data.

FIG. 10 is a block diagram showing the configuration of an ultrasonic diagnostic apparatus according to a second embodiment.

FIG. 11 is a flowchart showing operations of the ultrasonic diagnostic apparatus according to the second embodiment.

### DETAILED DESCRIPTION

In general, according to one embodiment, an ultrasonic probe includes a plurality of ultrasonic transducers and a calculation circuitry. The calculation circuitry calculates part of delay data relating to a delay time which is set in each of the ultrasonic transducers, before transmission of ultrasonic waves.

Hereinafter, an ultrasonic probe, an ultrasonic diagnostic apparatus, and an ultrasonic diagnostic assistance method according to the present application will be described with reference to drawings. In the embodiments described below, elements assigned with the same reference symbols perform the same operations, and redundant descriptions thereof will be omitted as appropriate.

### First Embodiment

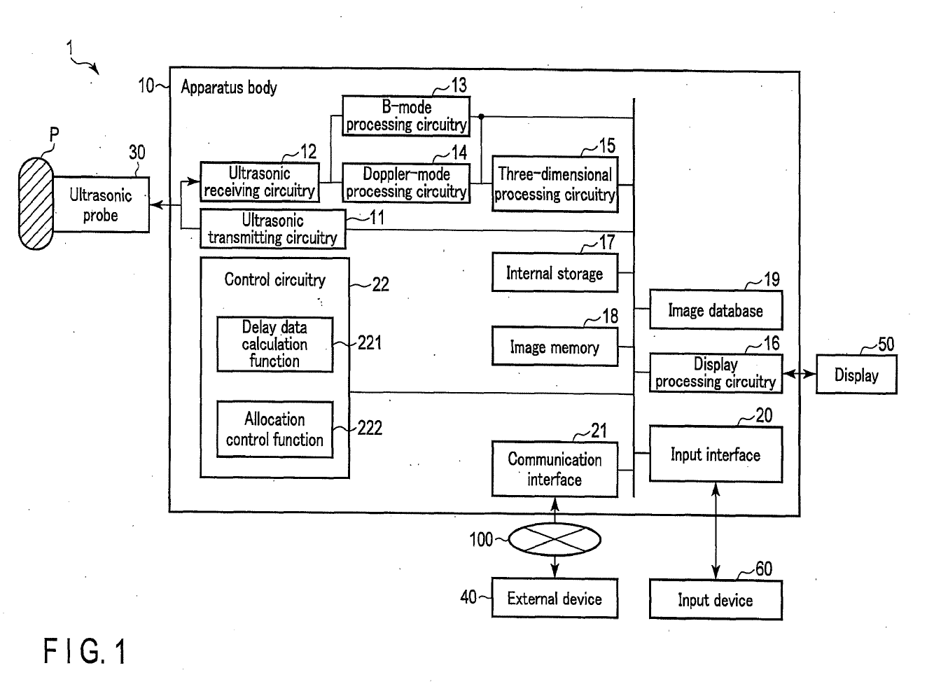

An ultrasonic diagnostic apparatus according to the present embodiment will be described with reference to the block diagram of FIG. 1.

FIG. 1 is a block diagram showing a configuration example of an ultrasonic diagram apparatus 1 according to the present embodiment. As shown in FIG. 1, the ultrasonic diagnostic apparatus 1 includes an apparatus body 10 and an ultrasonic probe 30. The apparatus body 10 is connected to an external device 40 via a network 100. The apparatus body 10 is connected to a display 50 and an input device 60.

The apparatus body 10 is an apparatus configured to generate an ultrasonic image based on echo received by the ultrasonic probe 30. The apparatus body 10 includes, as shown in FIG. 1, an ultrasonic transmitting circuitry 11, an ultrasonic receiving circuitry 12, a B-mode processing circuitry 13, a Doppler-mode processing circuitry 14, a three-dimensional processing circuitry 15, a display processing circuitry 16, an internal storage 17, an image memory 18 (cine memory), an image database 19, an input interface 20, a communication interface 21, and a control circuitry 22.

The ultrasonic transmitting circuitry 11 is a processor that supplies driving signals to the ultrasonic probe 30. The ultrasonic transmitting circuitry 11 is implemented, for example, by trigger generating circuitry, delay circuitry, and pulser circuitry, etc. The trigger generating circuitry repeatedly generates rate pulses for forming transmission ultrasonic waves at a predetermined rate frequency. The delay circuitry converges ultrasonic waves generated from the ultrasonic probe 30 as a beam, and applies, to each rate pulse generated by the trigger generating circuitry, a transmission delay time for each element required for determining a transmission directivity. The pulser circuitry supplies driving signals (a driving pulse) to the ultrasonic probe 30 at a timing based on the rate pulse. By changing the transmission delay time given to each rate pulse from the delay circuitry, the transmission direction from the element surface can be discretionally adjusted. The transmission delay time is calculated by a delay data calculation function 221 of the later-described control circuitry 22.

The ultrasonic receiving circuitry 12 is a processor that executes various processes on reflected wave signals received by the ultrasonic probe 30 to generate a receive signal. The ultrasonic receiving circuitry 12 is implemented, for example, by amplification circuitry, an A/D converter, reception delay circuitry, and an adder, etc. The amplification circuitry executes a gain correction process for each channel by amplifying reflected wave signals received by the ultrasonic probe 30. The A/D converter converts the gain-corrected reflected wave signals to digital signals. The reception delay circuitry delays input of the digital signals to the adder by a reception delay time required for determining a reception directivity. The adder adds a plurality of digital signals in which the reception delay time has been applied. After the addition process of the adder, receive signals are generated in which a reflected component from a direction corresponding to the reception directivity is emphasized. The reception delay time is calculated by a delay data calculation function 221 of the later-described control circuitry 22.

The B-mode processing circuitry 13 is a processor that generates B-mode data, based on the receive signals received from the ultrasonic receiving circuitry 12. The B-mode processing circuitry 13 executes an envelope detection process and a logarithmic amplification process, etc. on the receive signals received from the ultrasonic receiving circuitry 12, and generates data (B-mode data) in which the signal intensity is expressed by the brightness intensity. The generated B-mode data is stored in a RAW data memory (not shown in the drawings) as B-mode RAW data on an ultrasonic scanning line. The B-mode RAW data may be stored in the internal storage 17 described later.

The Doppler-mode processing circuitry 14 is a processor that generates a Doppler waveform and Doppler data, based on the receive signals received from the ultrasonic receiving circuitry 12. The Doppler-mode processing circuitry 14 extracts a blood flow signal from the receive signal, generates a Doppler waveform from the extracted blood flow signal, and generates data (Doppler data) in which information, such as a mean velocity, dispersion, power, etc. is extracted from the blood flow signal with respect to multiple points.

The three-dimensional processing circuitry 15 is a processor that can generate two-dimensional image data or three-dimensional image data (also referred to as volume data), based on the data generated by the B-mode processing circuitry 13 and the Doppler-mode processing circuitry 14. The three-dimensional processing circuitry 15 performs RAW-pixel conversion to generate two-dimensional image data consisting of pixels.

In addition, the three-dimensional processing circuitry 15 performs, to B-mode RAW data stored in a RAW data memory, RAW-voxel conversion which includes an interpolation process taking spatial position information into consideration to generate volume data consisting of voxels in a desired range. The three-dimensional processing circuitry 15 generates rendering image data by performing a rendering process to the generated volume data. The B-mode RAW data, the two-dimensional image data, the volume data, and the rendering image data are generically referred to as ultrasonic data.

The display processing circuitry 16 executes various processes, such as dynamic range, brightness, contrast and y curve corrections, and RGB conversion, etc. to image data generated in the three-dimensional processing circuitry 15, thereby converting the image data to a video signal. The display processing circuitry 16 directs the display 50 to display the video signal. The display processing circuitry 16 may generate a user interface (Graphical User Interface: GUI) through which an operator inputs various instructions by the input interface 20, and directs the display 50 to display the GUI. The display 50 may adopt, for example, a CRT display, a liquid crystal display, an organic EL display, an LED display, a plasma display, or any other displays known in this technical field.

The internal storage 17 includes, for example, a storage medium which is readable by a processor, such as a magnetic or optical storage medium, or a semiconductor memory, etc. The internal storage 17 stores a control program relating to a delay amount setting method, a control program for implementing ultrasonic transmission/reception, a control program for executing an image process, and a control program for executing a display process, according to the present embodiment. In addition, the internal storage 17 stores data constellation of diagnosis information (e.g., patient ID, doctor's findings, etc.), a diagnosis protocol, a body mark generation program, and data such as a conversion table for presetting a range of color data for use in imaging, with respect to each of regions of diagnosis. The internal storage 17 may store anatomical illustrations, for example, an atlas, relating to the structures of internal organs in the body.

In addition, the internal storage 17 stores two-dimensional image data, volume data, and rendering image data generated by the three-dimensional processing circuitry 15, in accordance with a storing operation input via the input interface 20. In accordance with a storing operation input via the input interface 20, the internal storage 17 may store two-dimensional image data, volume data, and rendering image data generated by the three-dimensional processing circuitry 15, along with an operation order and an operation time. The internal storage 17 can transfer the stored data to an external device through the communication interface 21.

The image memory 18 includes, for example, a storage medium which is readable by a processor, such as a magnetic or optical storage medium, or a semiconductor memory. The image memory 18 stores image data corresponding to a plurality of frames immediately before a freeze operation input through the input interface 20. The image data stored in the image memory 18 is successively displayed (cine-displayed), for example.

The image database 19 stores image data transferred from the external device 40. For example, the image database 19 acquires and stores past medical image data relating to a particular patient obtained by the past diagnosis and stored in the external device 40. The past medical image data includes ultrasonic image data, Computed Tomography (CT) image data, MR image data, Positron Emission Tomography (PET)-CT image data, PET-MR image data, and X-ray image data.

The image database 19 may store desired image data by reading image data stored in a storage medium such as an MO, a CD-R and a DVD.

The input interface 20 receives various instructions from an operator through the input device 60. The input device 60 is, for example, a mouse, a keyboard, a panel switch, a slider switch, a trackball, a rotary encoder, an operation panel, and a touch command screen (TCS). The input interface 20 is connected to the control circuitry 22, for example, via a bus. The interface 20 converts an operation instruction input by the operator into electric signals, and outputs the electric signals to the control circuitry 22. In the present embodiments, the input interface 20 is not limited to interface circuitry which is connected to physical operation components such as a mouse, a keyboard, etc. The input interface 20 may include processing circuitry of electric signals which receives, as radio signals, electric signals corresponding to an operation instruction input from an external input device independent from the ultrasonic diagnostic apparatus 1, and outputs the electric signals to the control circuitry 22. The external input device may be, for example, an external input device that is capable of transmitting an operation instruction corresponding to an instruction with a gesture by the operator as a wireless signal.

The communication interface 21 is connected to the external device 40 through the network 100, etc., and performs data communication with the external device 40. The external device 40 is, for example, a database of a picture archiving and communication system (PACS) which is a system for managing various medical image data, a database of an electronic medical record system for managing electronic medical records to which medical images are added, etc. In addition, the external device 40 may, for example, be any medical image diagnostic apparatus other than the ultrasonic diagnostic apparatus 1 according to the present embodiment, such as an X-ray CT apparatus, a magnetic resonance imaging (MRI) apparatus, a nuclear medical diagnostic apparatus, and an X-ray diagnostic apparatus, etc. Any standards may be applied for communication with the external device 40. For example, digital imaging and communication in medicine (DICOM) may be applied.

The control circuitry 22 is a processor acting as a nerve center of the ultrasonic diagnostic apparatus 1, for example. The control circuitry 22 executes a control program stored in the internal storage 17 to realize a function corresponding to the program. Specifically, the control circuitry 22 executes a delay data calculation function 221 and an allocation control function 222.

By executing the delay data calculation function 221, the control circuitry 22 calculates delay control data (delay data) relating to a delay time that has been set for each ultrasonic transducer (transmission delay time and reception delay time) during transmission or reception of an ultrasonic wave.

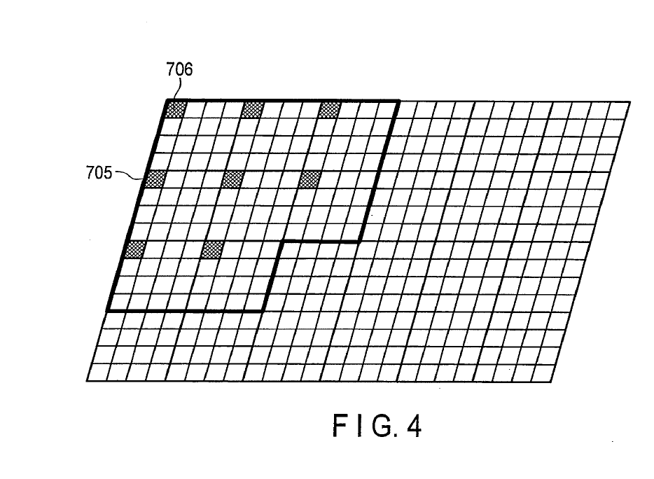

In the present embodiment, the apparatus body 10 shares with the ultrasonic probe 30 to perform the calculation process of delay data. That is, the ultrasonic probe 30 calculates part of delay data to be set, and the apparatus body 10 calculates remaining data of the delay data to be set. Details of the calculation process of delay data will be described layer with reference to FIGS. 3 and 4.

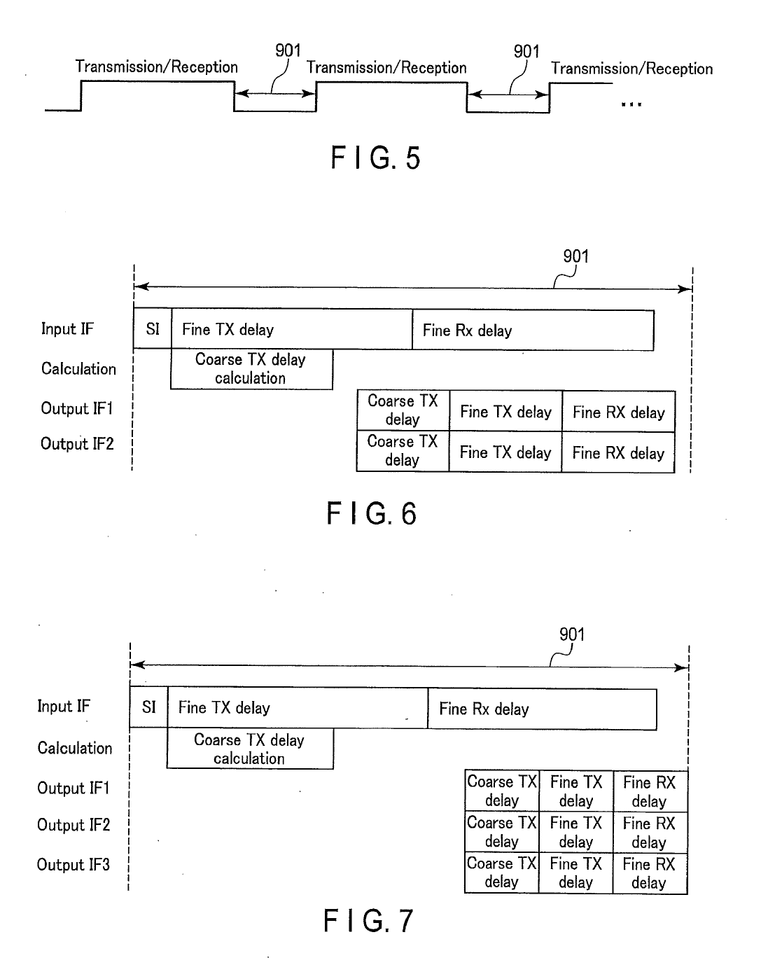

By executing the allocation control function 222, the control circuitry 22 allocates the calculation process of delay data to the delay data calculation function 221 and the ultrasonic probe 30 so that setting of a delay time to the ultrasonic probe is completed within a blanking period (referred to as a settable period) from an end of a reception period to the next transmission timing of ultrasonic waves. Allocation examples of the calculation process will be described with reference to FIGS. 5 to 9.

The delay data calculation function 221 and the allocation control function 222 may be incorporated as control programs, or specific hardware circuitries capable of executing each of the functions may be incorporated into the control circuitry 22 itself or the apparatus body 10.

The control circuitry 22 may be executed by an integrated circuit for specific purpose (Application Specific Integrated Circuit: ASIC) into which these exclusive hardware circuitries are integrated, Field Programmable Gate Array (FPGA), other complex programmable logic devices (CPLD), or simple programmable logic devices (SPLD).

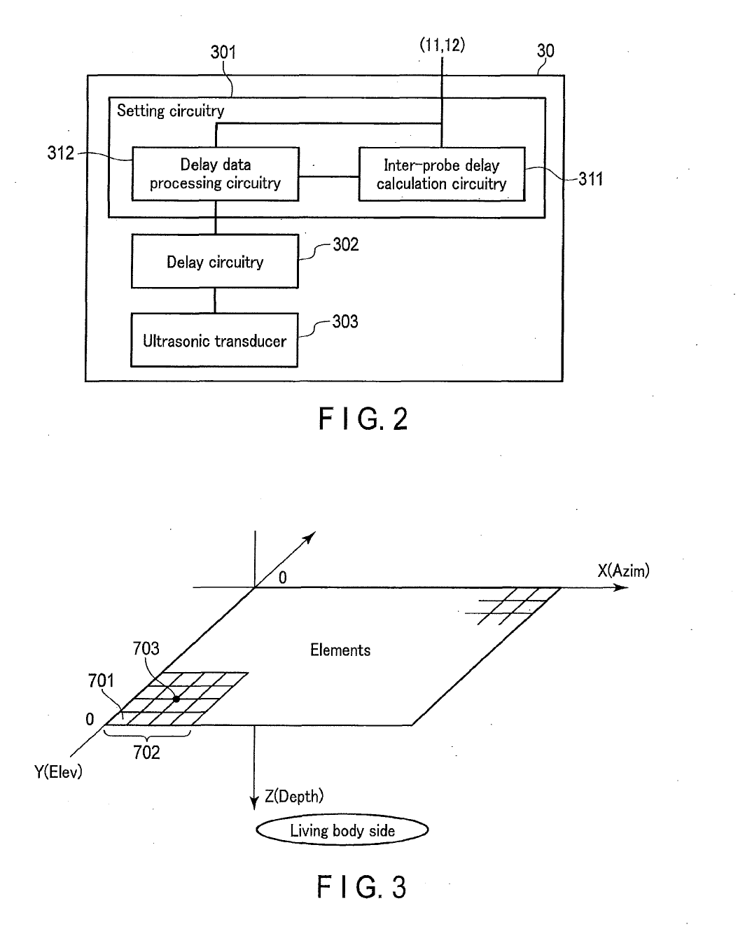

Next, the ultrasonic probe 30 according to the present embodiment will be described with reference to the block diagram of FIG. 2.

The ultrasonic probe 30 includes a setting circuitry 301, a delay circuitry 302, and a plurality of ultrasonic transducers 303 (simply, referred to as elements). The setting circuitry 301 includes an inter-probe delay calculation circuitry 311 and a delay data processing circuitry 312.

The ultrasonic probe 30 also includes a matching layer provided to elements and a backing material that prevents propagation of ultrasonic waves to the rear side of the elements, although they are not illustrated in the drawings. The ultrasonic probe 30 is detachably connected to the apparatus body 10.

The inter-probe delay calculation circuitry 311 receives setting information from the apparatus body 10 and calculates part of delay data using the setting information. The setting information is information before scanning by means of the ultrasonic probe 30, such as, focusing and the depth of an ultrasonic beam.

The delay data processing circuitry 312 receives part of data of the delay data that has been calculated by the inter-probe delay calculation circuit 311, and data in which calculation of remaining data of the delay data has been performed from the apparatus body 10, respectively. The delay data processing circuitry 312 transfers these delay data to the delay circuitry 302.

The delay circuitry 302 receives the delay data from the delay data processing circuitry 312 and sets a transmission delay time and a reception delay time for each sub-array and each of the elements belonging to a sub-array, based on the delay data.

The plurality of elements transmit ultrasonic waves generated based on driving signals to a living body P at timings corresponding to the delay time of each element set by the delay circuitry 302.

Once the ultrasonic waves are transmitted from the ultrasonic probe 30 to the living body P, the transmitted ultrasonic waves are sequentially reflected by the boundary showing discontinuity of the acoustic impedance of the living tissue of the living body P, and are received as reflected waves by the plurality of elements. The amplitude of the received reflected waves depend on the difference in the acoustic impedance at the boundary of the acoustic impedance on which the ultrasonic waves are reflected. If the transmitted ultrasonic pulses are reflected in a bloodstream or on the surface of the cardiac wall, the frequency of the reflected waves are shifted depending on velocity components in the direction of transmitting ultrasonic waves in a moving object due to the Doppler effect. The ultrasonic probe 30 receives the reflected waves from the living body P, converts the reflected waves into electrical signals, and transmits the electrical signals to the apparatus body 10.

### (Regarding Calculation Method of Each Delay Amount)

Hereinafter, cases of calculating three types of data are assumed. The three types of data are transmission delay data relating to each sub-array (also referred to as coarse transmission delay data), transmission delay data relating to each of elements belonging to a sub-array (also referred to as fine transmission delay data), and reception delay data relating to each of the elements (also referred to as fine reception delay data).

Calculation examples of the coarse transmission delay data, fine transmission delay data, and fine reception delay data will be described with reference to FIGS. 3 and 4.

FIG. 3 is a conceptual diagram showing an arrangement of two-dimensionally arrayed elements. In this figure, an azimuth direction is defined as an x direction, an elevation direction is defined as a y direction, and a depth direction is defined as a z direction. In the same figure, each cell corresponds to a two-dimensionally arrayed element position 701. Herein, it is defined that 4×4 elements belong to one sub-array 702. The center of the sub-array 702 is referred to as a sub-array position 703. A delay distance arises from the sub-array position 703 to a focus point for each sub-array 702.

In the present embodiment, the coordinates of a focus point in the living body P are defined as (xf, yf, zf), and the coordinates of the sub-array position 703 in the living body P are defined as (xs, ys, 0). A sub-array delay distance is calculated as coarse transmission delay data ds in accordance with equation (1).

ds=\sqrt{(xs−xf)²+(ys−yf)²+zf²}−\sqrt{xf²+yf²+zf²} (1)

The calculation of the equation (1) is performed for each sub-array 702.

A delay distance occurs for each element position 701 in the sub-array 702.

In the present embodiment, the coordinates of each element are defined as (xe, ye, 0). With respect to each of the elements belonging to the sub-array 702, a sub-array differential focus point distance is calculated as fine transmission delay data de in accordance with equation (2). Namely, a difference between a distance from the focus point to the element position 701 and a distance from the focus point to the sub-array position 703 is calculated.

de=\sqrt{(xe−xf)²+(ye−yf)²+zf²}−\sqrt{(xs−xf)²+(ys−yf)²+zf²} (1)

The sub-array differential focus point distance is calculated for each of the sub-arrays 702.

Next, one example of calculating the entire fine transmission delay data will be described with reference to FIG. 4.

For each area 705 (sub-array group) controlling a plurality of sub-arrays 702 in common, fine transmission delay data is calculated. Specifically, with respect to common same channel elements 706 (e.g., element positions on the oblique line) for each sub-array 702 in the area 705, a value at which the residual value of the sub-array differential focus point distance becomes a minimum is defined as fine transmission delay data of the same channel elements 706 in the area 705.

In the example of FIG. 4, eight sub-arrays 702 belong to one area 705, and thus eight sub-array differential focus point distances are calculated.

The fine reception delay data can be calculated basically by performing the same calculation as with the fine transmission delay data, and thus detailed descriptions thereof are omitted. To obtain fine reception delay data, the same value as with the fine transmission delay data may be used.

In transmission of ultrasonic waves, the delay circuitry 302 adds a delay based on the calculated coarse transmission delay data and fine transmission delay data to a driving signal from the apparatus body 10, and ultrasonic waves are generated from the elements. In reception of ultrasonic waves, a receive signal received from an element is amplified by a reception amplifier (unillustrated), the delay circuitry adds a delay based on fine reception delay data to the receive signal and outputs the result to an adding circuitry (unillustrated). In order to output the adding circuitry, a system delay (coarse reception delay) based on a sub-array delay is added at the ultrasonic receiving circuitry 12 in the apparatus body 10, and the result is further subjected to an adding process, thereby forming an ultrasonic beam.

(Transfer and Setting Processes of Delay Data)

Next, the transfer of delay data and setting process of delay data realized by the ultrasonic diagnostic apparatus 1 according to the present embodiment will be described.

First, the concept of the blanking period is shown in FIG. 5. The timing chart of the same figure shows transmission/reception intervals (Pulse Repetition Interval: PRI) of ultrasonic waves of the ultrasonic diagnostic apparatus 1 of the present embodiment.

The ultrasonic diagnostic apparatus 1 needs to finish a transfer process for delay data relating to ultrasonic waves that should be next transmitted and received from the apparatus body 10 to the ultrasonic probe 30 and a setting process of a delay time for each element in the ultrasonic probe 30 within a blanking period 901 shown in FIG. 5.

A first allocation example of the calculation process of the delay data to complete the transfer and setting processes within the blanking period 901 will be described with reference to FIG. 6.

FIG. 6 shows a sequence of data processing within the ultrasonic probe 30. The item “Input IF” indicates a transfer period of data which has been calculated at the control circuitry 22 of the apparatus body 10 and which will be input in the setting circuitry 301. The item “Calculation” indicates a calculation period in the inter-probe delay calculation circuitry 311. “Output IF1” and “Output IF2” each indicate a transfer period of data output from the setting circuitry 301 to the delay circuitry 302. The present embodiment assumes a case where the number of connections (also referred to as the number of lanes) of signal lines to perform data exchange between the setting circuitry 301 and the delay circuitry 302 is two, and thus two “Output IF” periods are shown. For the sake of convenience of explanation, it is assumed that upon delay data being output from “Output IF” to the delay circuitry 302, the setting process of the delay data is completed sequentially.

As shown in “Input IF” of FIG. 6, setting information (in the figure, SI), fine transmission delay data (in the figure, fine TX delay), and fine reception delay data (in the figure, fine RX delay) are input in this order. After completion of inputting of the setting information, an input of the fine transmission delay data is started in “Input IF”. Meanwhile, the inter-probe delay calculation circuit 311 starts calculation of delays relating to sub-arrays, i.e., coarse transmission delay data (in the figure, coarse TX delay), based on the setting information. In short, the ultrasonic probe 30 can calculate coarse transmission delay data while receiving fine transmission delay data.

From the viewpoint of the circuitry scale, the calculation amount of delay data in the inter-probe delay calculation circuitry 311 is desirably less than the calculation amount of delay data in the control circuitry 22 of the apparatus body 10. So, the control circuitry 22 that implements the allocation control function 222 allocates calculation amounts so that the inter-probe delay calculation circuit 311 calculates coarse transmission delay data which is a relatively small amount of calculation, and the delay data calculation function 221 of the apparatus body 10 calculates fine transmission delay data and fine reception delay data whose calculation amounts are larger than those of the coarse transmission delay data.

After completion of calculation of the coarse transmission delay data, the setting circuitry 301 outputs coarse transmission delay data, fine transmission delay data, and fine reception delay data in this order from “Output IF1” and “Output IF2” to the delay circuitry 302. The order of outputting delay data from Output IF is not limited to the sequential order shown in FIG. 6, and the fine transmission delay data, coarse transmission delay data and fine reception delay data may be output in this order.

Next, a second allocation example of the calculation process of delay data will be described with reference to FIG. 7.

In FIG. 7, the same transfer and setting processes as in the first allocation example shown in FIG. 6 is performed except that the number of lanes is different from the first allocation example shown in FIG. 6. If the number of lanes can be increased as shown in FIG. 7, transfer of delay data from the setting circuitry 301 to the delay circuitry 302 can be completed in a shorter amount of time. It is sufficient that the transfer of delay data to the delay circuitry 302 is completed within the blanking period, and thus as the outputting timings in Outputs IF1 to IF3, the delay data is transmitted sequentially upon completion of the calculation of the coarse transmission delay data at the inter-probe delay calculation circuitry 311.

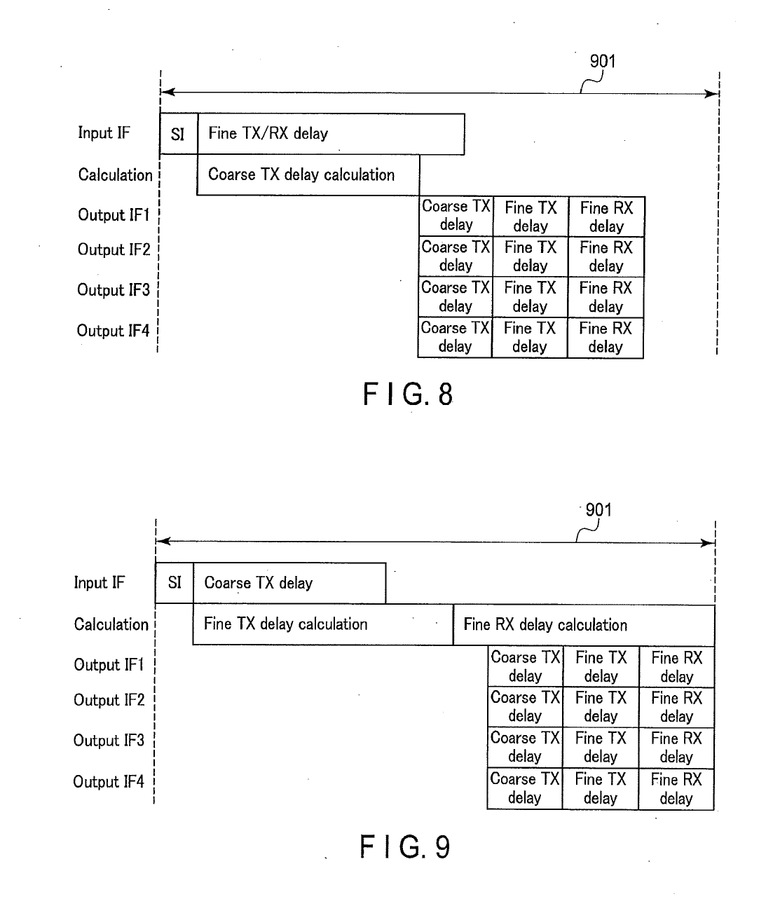

Next, a third allocation example of the calculation process of delay data will be described with reference to FIG. 8.

The example shown in FIG. 8 illustrates a case where the number of lanes is four, and the value of fine transmission delay data is the same as the value of fine reception delay data. In the case where fine transmission delay data and fine reception delay data have the same value, either the fine transmission delay data or the fine reception delay data is received from the apparatus body 10.

Next, a fourth allocation example of the calculation process of delay data will be described with reference to FIG. 9.

It is considered that if scanning by an ultrasonic probe is not executed, such as a case where a so-called freeze operation is performed, a time of several hundred milliseconds is given as a blanking period. In such a case, it is possible to take some time for the calculation process of delay data.

In such a case, the inter-probe delay calculation circuitry 311 may perform operations other than the calculation of coarse transmission delay data, provided that the calculation and setting processes of delay data are completed within a blanking period 901. For example, as shown in FIG. 9, the setting information and the coarse transmission delay data transferred from the apparatus body 10 is input in the delay data processing circuitry 312. The inter-probe delay calculation circuitry 311 calculates fine transmission delay data and fine reception delay data. The delay data processing circuitry 312 transfers the coarse transmission delay data received from the apparatus body and the fine transmission delay data and fine reception delay data calculated at the inter-probe delay calculation circuitry 311 to the delay circuitry 302.

If the condition that the calculation and setting processes of delay data are complete within the blanking period 901 is satisfied, the inter-probe delay calculation circuit 311 may calculate part of the fine transmission delay data and fine reception delay data, in addition to the calculation of the coarse transmission delay data.

In the above descriptions, it is explained that the allocation control function 222 allocates the calculation process of delay data to the delay data calculation function 221 and the ultrasonic probe 30; however, the allocation control function 222 may not necessarily be provided. For example, the ultrasonic diagnostic apparatus may be preliminarily set so that the coarse transmission delay data is calculated by the inter-probe delay calculation circuitry 311, and the fine transmission delay data and fine reception delay data is calculated by the delay data calculation function 221 of the apparatus body 10.

According to the first embodiment described above, separately from the delay calculation function of the apparatus body, the ultrasonic diagnostic apparatus includes a delay calculation circuitry in the ultrasonic probe, and shares calculation of delay data necessary for delay setting, based on the condition that setting of the delay data is completed within a blanking period. In the ultrasonic probe, part of delay data is calculated in the ultrasonic prove during a time when the delay data calculated in the apparatus body is transferred. It is possible to reduce the transfer amount of delay data from the apparatus body, and even if the number of elements (the number of channels) of a two-dimensional array probe is increased, it is possible to transfer and set the delay data within a desired blanking period, without increasing a clock frequency of input IF of the ultrasonic probe.

### Second Embodiment

The first embodiment is predicated upon connecting the ultrasonic probe 30 preliminarily including a calculation circuitry (inter-probe delay calculation circuitry 311); however, a case is also assumed where an ultrasonic probe having the number of elements that does not require sharing delay data between an apparatus body and an ultrasonic probe to perform calculation is connected.

Therefore, in the second embodiment, flexible control can be realized by checking the type etc. of a connected ultrasonic probe and determining whether to allocate a calculation process of delay data to the ultrasonic probe based on the determined result.

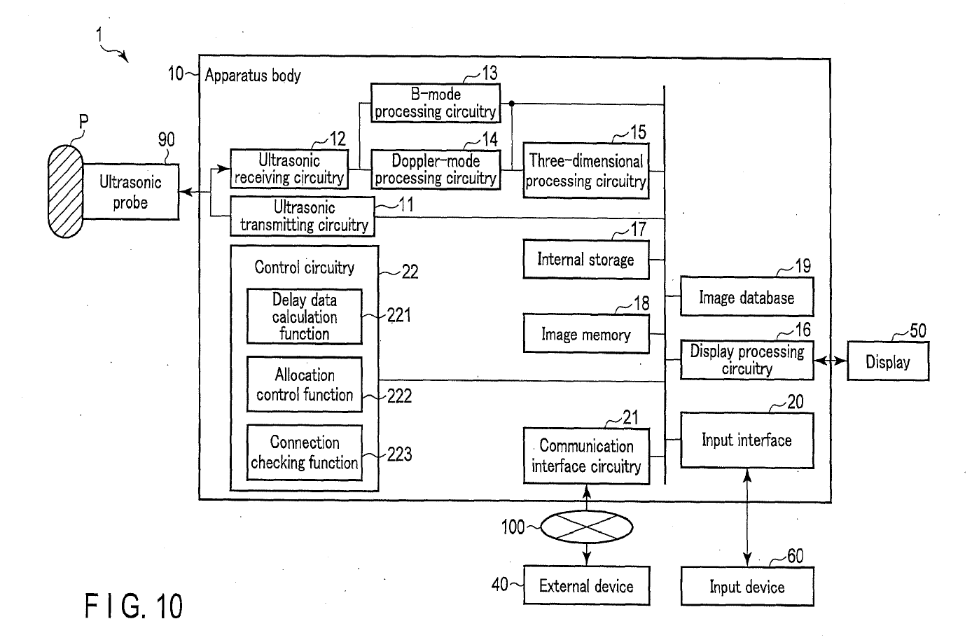

The block diagram of an ultrasonic diagnostic apparatus 1 according to the second embodiment is shown in FIG. 10.

The ultrasonic diagnostic apparatus 1 according to the second embodiment has the same configuration as the first embodiment except that a control circuit 22 further executes a connection checking function 223 in addition to the functions according to the first embodiment.

By executing the connection checking function 223, the control circuitry 22 checks the type of a connected ultrasonic probe 90 to determine whether to perform allocation control of a calculation process of delay data. The control circuitry 22 may determine the number of elements of the ultrasonic array probe by executing the connection checking function 223.

The connection checking function 223 may be incorporated as a control program, or specific hardware circuitry capable of executing each of the functions may be incorporated into the control circuitry 22 itself or the apparatus body 10.

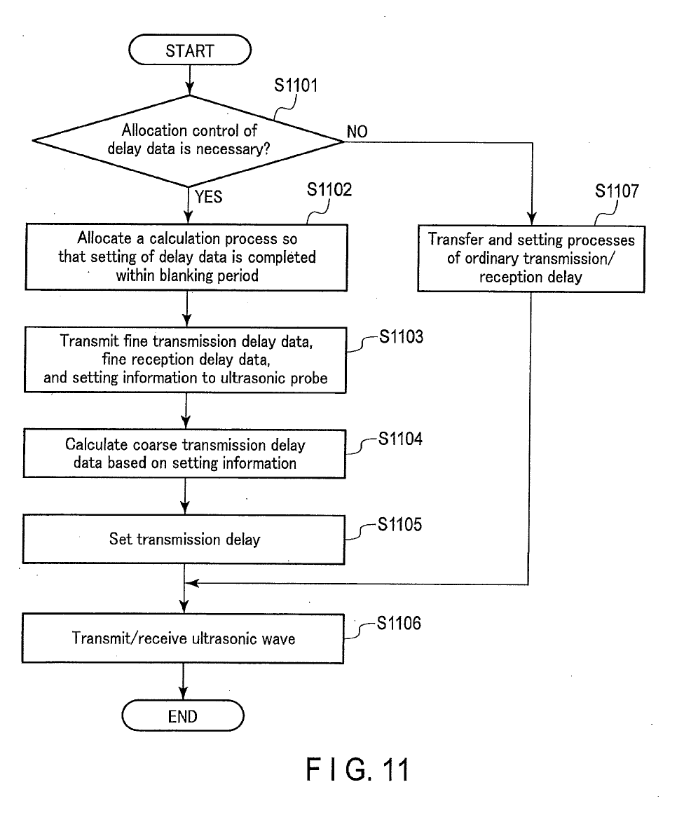

Next, the operation of the ultrasonic diagnostic apparatus 1 according to the second embodiment will be explained with reference to a flowchart of FIG. 11.

In step S1101, the control circuitry 22 that executes the connection checking function 223 determines whether or not the connected ultrasonic probe performs allocation control of the calculation process of delay data. The determination whether to perform the allocation control is made as follows, for example. It is determined that the ultrasonic probe 90 will perform the allocation control if the ultrasonic probe 90 includes inside the inter-probe delay calculation circuitry 311, and it is determined that the ultrasonic probe will perform the allocation control if the number of elements is equal to or more than a threshold. These determinations may be made by the control circuitry 22 that executes the connection checking function 223 based on the following production information, for example, by configuring so that production information relating to the specification of the ultrasonic probe 90 (presence or absence of a calculation circuitry, the number of elements, etc.) is preliminarily prepared, and the product information is transmitted when the ultrasonic probe 90 is connected. If the allocation control is performed, the process proceeds to step S1102, and if the allocation control is unnecessary, the process proceeds to step S1107.

In step S1102, the control circuitry 22 allocates the calculation process in accordance with the calculation amounts of delay data so that setting of the delay data is completed within the blanking period. For example, the data amounts of the coarse transmission delay data, fine transmission delay data and fine reception delay data to be calculated can be grasped in accordance with the number of elements, and a time period necessary for the calculation can be estimated based on the data amounts and the clock frequency. Therefore, the calculation process may be allocated in accordance with the processability of the inter-probe delay calculation circuitry 311 and the delay data calculation function 221 so that the transfer and setting processes of delay data are completed within the blanking period.

In step S1103, the apparatus body 10 transmits fine transmission delay data, fine reception delay data, and setting information that have been preliminarily calculated by the delay data calculation function 221 to the ultrasonic probe.

In step S1104, the inter-probe delay calculation circuitry 311 calculates coarse transmission delay data based on the setting information.

In step S1105, the setting circuitry 301 transfers the coarse transmission delay data, fine transmission delay data, and reception delay data to the delay circuitry 302, thereby the delay data being set.

In step S1106, the delay circuitry 302 adds a delay to a driving signal based on each delay data, and transmits and receives ultrasonic waves.

In step S1107, an ultrasonic probe that does not require allocation control is connected, and thus the apparatus body 10 may calculate ordinary transmission/reception delay data, and the apparatus body 10 may perform the transfer and setting processes of transmission/reception delay data. Thereafter, the process proceeds to step S1106 to perform transmission and reception of ultrasonic waves.

According to the second embodiment described above, it is possible to complete transfer and setting processes of delay data within a blanking period in the same manner as in the first embodiment and realize flexible control by checking the type of a connected ultrasonic probe and performing allocation control of the calculation process of delay data based on the determined result.

In the embodiment described above, as an ultrasonic probe, a two-dimensional array probe including a plurality of two-dimensionally arrayed elements is described by way of example. However, the ultrasonic probe according to the present embodiment is not limited thereto, and even if it is an ultrasonic probe including a plurality of one-dimensionally arrayed elements (also referred to as one-dimensional array probe), the ultrasonic probe may perform the above-mentioned allocation control of a calculation process of delay data.

The number of one-dimensionally arrayed elements is considered to be less than the number of two-dimensionally arrayed elements; however, when the blanking period is extremely short, or when the frame rate is high and the data volume is extremely large, the above-mentioned allocation control of a calculation process of delay data needs to be performed in such a one-dimensional array probe.

In the case of a one-dimensional array probe, for example, considering transmission delay data relating to a group of a plurality of elements as coarse transmission delay data, and considering transmission delay data relating to each element of said group of elements as fine transmission delay data, the allocation control of the calculation process is performed.

The functions described in connection with the above embodiments may be implemented, for example, by installing a program for executing the processing in a computer, such as a work station, etc., and expanding the program in a memory. The program that causes the computer to execute the processing can be stored and distributed by means of a storage medium, such as a magnetic disk (a hard disk, etc.), an optical disk (CD-ROM, DVD, Blu-ray® Disc, etc.), and a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic probe comprising:
a plurality of ultrasonic transducers; and
a calculation circuitry that calculates part of delay data relating to a delay time which is set in each of the ultrasonic transducers, before transmission of ultrasonic waves.

2. An ultrasonic diagnostic apparatus comprising:
an ultrasonic probe which comprises:
a plurality of ultrasonic transducers; and
a calculation circuitry that calculates part of delay data relating to a delay time which is set in each of the ultrasonic transducers, before transmission of ultrasonic waves; and
a processor that calculates remaining delay data of the delay data.

3. The apparatus according to claim 2, wherein the processor allocates a calculation process to the calculation circuitry and the processor so that setting of the delay time to the ultrasonic probe is completed within a blanking period from an end of a reception period of the ultrasonic waves to a next transmission timing of ultrasonic waves.

4. The apparatus according to claim 3, wherein the processor allocates, to the calculation circuitry, a calculation amount which is less than a calculation amount allocated for the processor.

5. The apparatus according to claim 2, wherein the calculation circuitry calculates coarse transmission delay data relating to a sub-array, and the processor calculates fine transmission delay data relating to each of the ultrasonic transducers belonging to the sub-array.

6. The apparatus according to claim 3, wherein the processor determines whether or not to perform allocation of the calculation process of delay data in accordance with a type of a connected ultrasonic probe.

7. An ultrasonic diagnostic assistance method comprising:
allocating a calculation process of delay data relating to a delay time which is set in each of ultrasonic transducers to a calculation circuitry included in an ultrasonic probe and an ultrasonic diagnostic apparatus body, before transmission and reception of ultrasonic waves,
causing the calculation circuitry of the ultrasonic probe to calculate part of the delay data, and
causing the ultrasonic diagnostic apparatus body to calculate remaining data of the delay data.
